(19) United States
(12) Patent Application Publication
(10) Pub. No.: US 2024/0065563 A1
(43) Pub. Date: Feb. 29, 2024

(54) BLOOD PRESSURE MEASUREMENTS USING CYCLIC PRESSURE ON A WEARABLE DEVICE PPG SENSOR

(71) Applicant: ChroniSense Medical Ltd., Yokneam (IL)

(72) Inventor: Daniel H. Lange, Kfar Vradim (IL)

(21) Appl. No.: 18/498,876

(22) Filed: Oct. 31, 2023

Related U.S. Application Data
(63) Continuation-in-part of application No. 17/463,284, filed on Aug. 31, 2021.
Application No. 17/463,284 is a continuation-in-part of application No. 15/226,881, filed on Aug. 2, 2016, now Pat. No. 11,160,461.
Application No. 15/226,881 is a continuation-in-part of application No. 14/738,666, filed on Jun. 12, 2015, now Pat. No. 11,160,459.
Application No. 14/738,666 is a continuation-in-part of application No. 14/738,636, filed on Jun. 12, 2015, now Pat. No. 11,712,190.
Application No. 14/738,636 is a continuation-in-part of application No. 14/738,711, filed on Jun. 12, 2015, now Pat. No. 10,470,692.

(51) Int. Cl. A61B 5/0225; A61B 5/00; A61B 5/021; A61B 5/107

(52) U.S. Cl. CPC A61B 5/02255; A61B 5/02125; A61B 5/1075; A61B 5/7278; A61B 5/0245

(57) ABSTRACT

Systems and methods for blood pressure measurement (BP) based on photoplethysmogram (PPG) data and electrocardiogram (ECG) data an initial baseline BP from a reliable source. A force is cyclically applied to a PPG sensor and the PPG pulse data is recorded and analyzed to determine a PPG-pulse-peak value. The initial baseline MAP is used to configure a MAP model. Additionally, a pre-determined PTT model utilizing PPG and ECG data along blood vessel parameters and baseline BP are initialized to generate a BP. Subsequent BP measurements use the same process of determining PPG-pulse-peak values, the initialize MAP model and pre-determined PTT model to determine a new BP value.

### CROSS-REFERENCE TO RELATED APPLICATIONS

The present application is a Continuation in Part of U.S. patent application Ser. No. 17/463,284, titled “Blood Pressure Measurement Using a Wearable Device”, filed on Aug. 31, 2021. The application Ser. No. 17/463,284 is a Continuation in Part of U.S. patent application Ser. No. 15/226,881, titled “Blood Pressure Measurement Using a Wearable Device”, filed on Aug. 2, 2016, now U.S. Pat. No. 11,160,461. The application Ser. No. 15/226,881 is a Continuation in Part of U.S. patent application Ser. No. 14/738,666, titled “Monitoring Health Status of People Suffering from Chronic Diseases,” filed on Jun. 12, 2015, now U.S. Pat. No. 11,160,459 and is a Continuation in Part of U.S. patent application Ser. No. 14/738,636, titled “Wearable Device Electrocardiogram,” filed on Jun. 12, 2015, now U.S. Pat. No. 11,712,190, and is also a Continuation in Part of U.S. patent application Ser. No. 14/738,711, titled “Pulse Oximetry,” filed on Jun. 12, 2015, now U.S. Pat. No. 10,470,692. The disclosures of the aforementioned applications are incorporated herein by reference for all purposes.

### FIELD

The present application relates to systems and methods for monitoring the health status of people, more specifically, to systems and methods for optimizing sensor pressure in continuous or intermittent non-invasive blood pressure (NIBP) measurements using wearable devices using a photoplethysmogram (PPG) sensor, an electrocardiogram (ECG) data using input plates, and an initial reliable baseline measurement.

### BACKGROUND

It should not be assumed that any of the approaches described in this section qualify as prior art merely by virtue of their inclusion in this section.

Blood pressure (BP) is one of the basic medical parameters used to diagnose human health condition. The most accurate methods for BP measurements involve insertion of a catheter into a human artery. However, the BP measurements using a catheter are invasive and costly since they require a medical professional to perform the measurements and, typically, can only be performed in a medical facility environment.

Less accurate methods for BP measurements include use of an inflatable cuff to pressurize a blood artery. There are numerous cuff-based portable devices for BP measurements that patients can use at home and do not require the assistance of a medical professional. However, cuff-based measurements require inflation and deflation of the inflatable cuff. Therefore, such devices are cumbersome to use and not suitable for ongoing BP measurements.

Some cuff-less devices for BP measurements use an electrical sensor to measure an electrocardiogram (ECG) and optical sensors to measure a photoplethysmogram (PPG). The ECG and PPG can be analyzed to determine pulse transit time (PTT). Because the PTT is in-part inversely proportional to the BP, the BP can in some cases be determined from the PTT using a pre-defined relationship. However, changes in a cardio-vascular status of a patient require often re-calibration of PTT based blood pressure measurements. Cuff-less devices can potentially provide continuous monitoring of the BP while imposing a minimal burden on normal activities when worn on various body parts such as a finger, a wrist, or an ankle.

Determining the BP based on the PTT alone may not be sufficiently accurate because of other cardiovascular parameters affecting hemodynamics such as vascular resistance, cardiac output, pulse rate (PR), temperature of a finger (if PPG is measured at the finger), and so forth. To compensate for influences of other parameters, some existing techniques for measuring of BP using the PPG include applying correction factors to account for the vascular resistance and age of patient. The correction factors can be determined by an empirical formula. Some other techniques attempt to determine compensation factors to compensate for various additional influences (for example, contacting force to sensors, nervous activity and cardiac output of patient, and ambient temperature). The compensation factors can be determined using a calibration process.

However, all currently known methods for cuff-less, non-inflatable BP or NIBP monitoring require frequent re-calibration to compensate for unaccounted changes in the cardiovascular status of a patient. Moreover, in the PTT and BP measurements carried out using wearable devices, the accuracy of the PTT and BP depends on the pressure that sensors of the wearable device apply to the skin of patient and location of the sensors with respect to blood vessels of a patient. Because the pressure and the location of the sensors change each time the patient puts the wearable device on or corrects location of the wearable device on their body, the corresponding re-calibration would be also required to account for change in the pressure and the location of the sensors. Therefore, there is a need for an NIBP monitoring that is less sensitive to changes in the pressure and location of the sensors without frequent re-calibrations.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

According to one aspect of the present disclosure, systems and methods are provided for blood pressure (BP) measurement based on photoplethysmogram (PPG) data and electrocardiogram (ECG) data from a wearable device worn with the PPG sensor substantially located over an artery. The method and system include an initialization phase and can include setting parameters in a MAP model and the normalization of parameters in a pre-determined model utilizing the pulse-transmission-time (PTT) derived from the ECG and a derived pulse rate (PR) from PPG data. The initialization phase includes generating a baseline systolic, diastolic, and MAP (mean arterial pressure) value using a highly reliable device, including but not limited to a sphygmomanometer.

The PPG-pulse-peak value at the apex pressure point needs to be determined to initialize the MAP model and can be part of the parameters of the pre-determined model for PTT based blood pressure measurements. To determine the PPG-pulse-peak value at the apex pressure point, the PPG data is recorded as the pressure against the PPG sensor, located against an artery, is increased and decreased over one or more cycles. The PPG peaks at the apex pressure point are determined using curve fitting to determine an apex of the PPG-pulse-peak values. The net pressure applied onto the arterial wall at the apex pressure point corresponds to the MAP pressure. Signal analysis can determine a PPG-pulse-peak value at the apex pressure point for each cycle. These can be averaged together to provide an estimate of the PPG-pulse-peak value.

The baseline systolic, diastolic, and MAP data is used to initialize a MAP model or MAP equation for conversion of the PPG-pulse-peak value into a MAP value. The MAP can be used in conjunction with or as part of the generation of a BP using a pre-determined model based on PTT and parameters that include changes in the diameter of the blood artery.

After initialization of the MAP model and the pre-defined model based on the PTT and PR data, the method and system can wait for an indication to take another BP measurement. Alternatively, the system and method can continually determine and report BP measurements. Upon receiving an indication for taking a BP measurement, further-PPG data and further ECG data are recorded in response to gradually increasing and decreasing pressure over one or more cycles. The further PPG and further ECG data are analyzed to determine one or more PTT value and a BP utilizing regression of a previously normalized pre-determined model. Additionally, the further PPG data is analyzed to determine PPG-peak values. Using the MAP model, a new-MAP value is generated from the PPG-pulse-peak values.

According to another example embodiment of the present disclosure, the steps of the method for optimizing sensor pressure in blood pressure measurements using a wearable device are stored on a non-transitory machine-readable medium comprising instructions, which, when implemented by one or more processors, perform the above recited steps.

Other example embodiments of the disclosure and aspects will become apparent from the following description taken in conjunction with the following drawings.

### BRIEF DESCRIPTION OF THE DRAWINGS

Embodiments are illustrated by way of example and not by limitation in the figures of the accompanying drawings, in which like references indicate similar elements.

FIG. 1 is a block diagram showing an example system for performing a blood pressure measurement using a wearable device.

FIG. 2 is a block diagram showing components of an example device for performing blood pressure measurement.

FIG. 3 is block diagram illustrating an example device for measuring arterial blood pressure at a wrist.

FIG. 4 shows an example plot of an ECG and an example plot of a PPG.

FIG. 5 shows an example plot of a PPG and an example plot of a blood vessel diameter.

FIG. 6 is a flow chart showing an example method for performing blood pressure measurements.

FIG. 7 shows a diagrammatic representation of a computing device for a machine, within which a set of instructions for causing the machine to perform any one or more of the methodologies discussed herein can be executed.

FIG. 8A is a diagrammatic representation of a blood vessel and optical sensor(s).

FIG. 8B is a plot of a compliance of a blood vessel, according to an example embodiment.

FIG. 9 shows plots of PPGs measured at different values of a sensor pressure, according to an example embodiment.

FIG. 10 is a flow chart showing an example method for optimizing sensor pressure in blood pressure measurements.

FIG. 11 is a block diagram showing an example system for performing a blood pressure measurement using a wearable device.

FIG. 12 shows a plot of PPG and MAP measurements and a curve fitted through an estimated MAP point and estimated diastolic and systolic pressure points.

FIGS. 13A, 13B and 13C are a flow chart showing a method for generating an estimated diastolic and systolic blood pressure and MAP value.

FIG. 14 shows a plot of an experiment showing a PPG signal as the pressure on the PPG sensor increases.

FIG. 15 shows a plot of an experiment showing a PPG envelope signal-to-noise ratio over time as the pressure on the PPG sensor increases.

### DETAILED DESCRIPTION

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical and electrical changes can be made without departing from the scope of what is claimed. The following detailed description is, therefore, not to be taken in a limiting sense, and the scope is defined by the appended claims and their equivalents.

The present disclosure provides systems and methods for performing BP measurement. Embodiments of the present disclosure allow for continuous or intermittent measuring of blood pressure of a patient in a non-intrusive manner while, for example, the patient is at home, at work, outdoors, traveling, or located at some other stationary or mobile environment. Embodiments of the present disclosure include a wearable device. The wearable device can be worn at a wrist, ankle, chest, neck, or positioned at other sites on a human body. The wearable device can allow measuring blood pressure of the patient without requiring the patient to take an active role in the process. The blood pressure data collected over an extended period of time can be analyzed to detect and track trends in medical parameters and to make conclusions concerning symptoms and progression of one or more chronic diseases from which the patient may suffer.

Some embodiments of the present disclosure can allow optimizing sensor pressure in BP measurements to increase accuracy of determination of the BP. According to some example embodiments, a method for optimizing sensor pressure in BP measurements using a wearable device may include recording, by at least one processor, photoplethysmogram (PPG) data using a PPG sensor of a wearable device while a pressure applied by the PPG sensor to a blood artery of a user is gradually increasing. The method may include monitoring, by the processor, a pulsating parameter associated with the PPG data. The pulsating parameter may change in response to the gradually increasing pressure. The method may include determining, by the processor, that the pulsating parameter has passed a critical value. In response to the determination, the method may include causing, by the processor, the increase of the pressure to stop. The method may include recording, by the processor, further PPG data using the PPG sensor and electrocardiogram (ECG) data using input plates of the wearable device. The method may include analyzing, by the processor, the further PPG data and the ECG data to determine a pulse transit time (PTT), a pulse rate (PR), and a diameter parameter. The diameter parameter may include a change in the diameter of the blood artery. The method may include determining, by the at least one processor and using a pre-defined model, a BP based on the PTT, the PR, and the diameter parameter. The pre-defined model can establish a relationship between the PTT, the PR, the diameter parameter, and the BP.

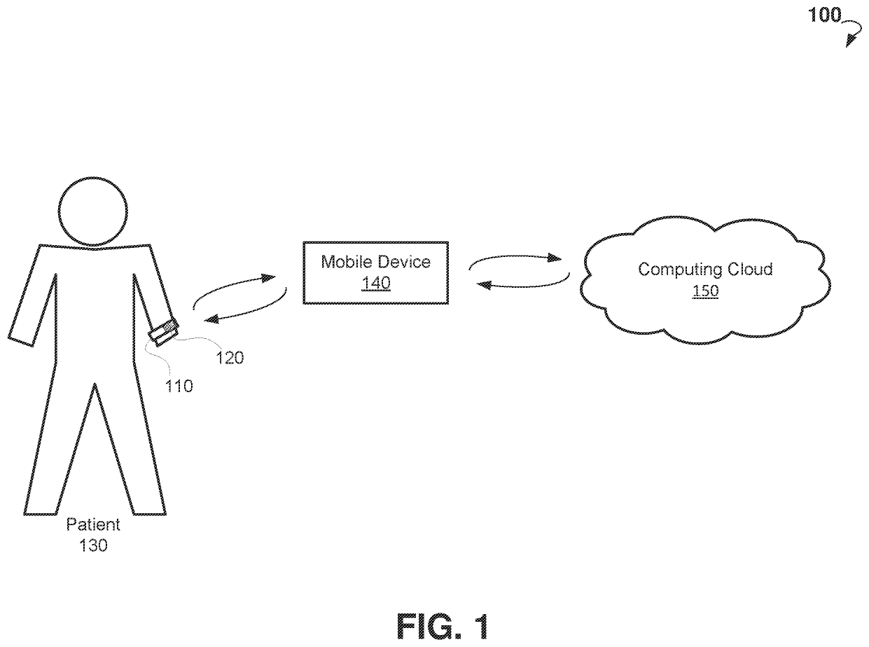

Referring now to FIG. 1, an example system 100 for performing blood pressure measurements is shown. The system 100 can include at least a wearable device 110. The wearable device 110 can include sensors 120. In some embodiments, the wearable device 110 is worn by a patient 130 (for example, on a wrist, ankle, earlobe, neck, chest, fingertip, and the like) for an extended period of time. In various embodiments, the wearable device 110 can be carried out as a watch, a bracelet, a wristband, a belt, a neck band, and the like.

The wearable device 110 can be operable to constantly collect, via sensors 120, sensor data from a patient 130. Based on the sensor data, the wearable device 110 can be operable to provide PPG and ECG data. The PPG and ECG data can be further used to obtain further medical parameters (for example, pulse rate, pulse transition time, blood pressure, and so forth).

In some embodiments, the system 100 includes a mobile device 140. The mobile device 140 can be communicatively coupled to the wearable device 110. In various embodiments, the mobile device 140 is operable to communicate with the wearable device 110 via a wireless connection such as, for example, Wi-Fi, Bluetooth, Infrared (IR), and the like. The mobile device 140 can include a mobile phone, a smart phone, a phablet, a tablet computer, a notebook, and so forth. The mobile device 140 can be operable to receive the sensor data and analyze the sensor data to provide ECG and PPG.

In further embodiments, the system 100 may include a cloud-based computing resource (also referred to as a computing cloud) 150. In some embodiments, the computing cloud 150 includes one or more server farms/clusters comprising a collection of computer servers and is co-located with network switches and/or routers. In certain embodiments, the mobile device 140 is communicatively coupled to the computing cloud 150. The mobile device 140 can be operable to send the sensor data to the computing cloud 150 for further analysis (for example, for extracting medical parameters from the ECG and PPG and storing the results). The computing cloud 150 can be operable to run one or more applications and to provide reports regarding a health status of the patient, based on trends in medical parameters over time.

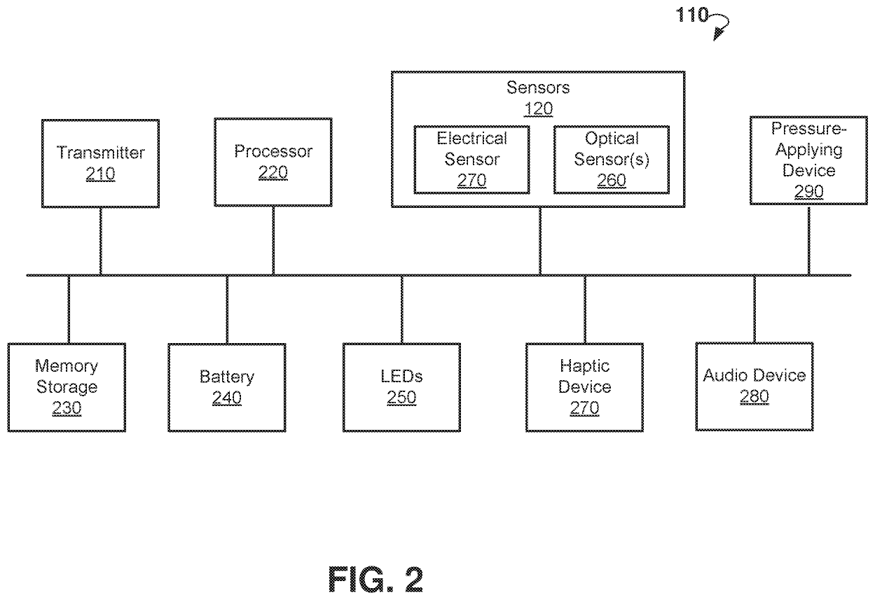

FIG. 2 is a block diagram illustrating components of wearable device 110, according to an example embodiment. The example wearable device 110 includes a transmitter 210, a processor 220, memory storage 230, a battery 240, light-emitting diodes (LEDs) 250, optical sensor(s) 260, electrical sensor 270, a haptic device 270, an audio device 280, and a pressure-applying device 290. The wearable device 110 may comprise additional or different components to provide a particular operation or functionality. Similarly, in other embodiments, the wearable device 110 includes fewer components that perform similar or equivalent functions to those depicted in FIG. 2.

The transmitter 210 can be configured to communicate with a network such as the Internet, a Wide Area Network (WAN), a Local Area Network (LAN), a cellular network, and so forth, to send data streams (for example sensor data, PPG data, and messages).

The processor 220 can include hardware and/or software, which is operable to execute computer programs stored in memory 230. The processor 220 can use floating point operations, complex operations, and other operations, including processing and analyzing data obtained from electrical sensor 270 and optical sensor(s) 260.

In some embodiments, the battery 240 is operable to provide electrical power for operation of other components of the wearable device 110. In some embodiments, the battery 240 is a rechargeable battery. In certain embodiments, the battery 240 is recharged using an inductive charging technology.

In various embodiments, the LEDs 250 are operable to emit light signals. The light signals can be of a red wavelength (typically 660 nm) or infrared wavelength (660 nm). Each of the LEDs 250 is activated separately and accompanied by a “dark” period where neither of the LEDs 250 is on to obtain ambient light levels. In some embodiments, a single LED 250 can be used to emit both the infrared and red-light signals. The lights can be absorbed by human blood (mostly by hemoglobin). The oxygenated hemoglobin absorbs more infrared light while deoxygenated hemoglobin absorbs more red light. Oxygenated hemoglobin allows more red light to pass through, while deoxygenated hemoglobin allows more infrared light to pass through. In some embodiments of the present disclosure, the LEDs 250 are also operable to emit light signals of isosbestic wavelengths (typically 810 nm and 520 nm). Both oxygenated hemoglobin and deoxygenated hemoglobin absorb the light of the isosbestic wavelengths equally.

The optical sensor(s) 260 (typically a photodiode) can receive light signals modulated by human tissue. Intensity of the modulated light signal represents a PPG. Based on the changes in the intensities of the modulated light signals, one or more medical parameters, such as, for example, oxygen saturation, arterial blood flow, pulse rate, and respiration, can be determined.

The LEDs 250 and optical sensor(s) 260 can be utilized in either a transmission or a reflectance mode for pulse oximetry. In the transmission mode, the LEDs 250 and optical sensor(s) 260 are typically attached or clipped to a translucent body part (e.g., a finger, toe, and earlobe). The LEDs 250 are located on one side of the body part while the optical sensor(s) 260 are located directly on the opposite site. The light passes through the entirety of the body part, from one side to the other, and is thus modulated by the pulsating arterial blood flow. In the reflectance mode, the LEDs 250 and optical sensor(s) 260 are located on the same side of the body part (e.g. a forehead, a finger, and a wrist), and the light is reflected from the skin and underlying near-surface tissues back to the optical sensor(s) 260.

The haptic device 270 can be configured to provide the patient a haptic feedback. For example, the haptic device may include a tap-in device, to apply a force or vibration to skin of the patient.

The audio device 280 can be configured to provide the patient a sound feedback. The audio device 280 can include a beeper configured to generate sounds of one or more pre-determined wavelengths.

The pressure-applying device 290 may be configured to apply external pressure to the optical sensor(s) 260 to force the optical sensor(s) 260 to contact the skin of a patient with different values of a contact force. In some embodiments, the pressure-applying device 290 may include an electrical motor and a spring touching the optical sensor(s) 260. The electrical motor can be configured to stretch the spring gradually causing the optical sensor(s) 260 to apply gradually increasing pressure to the skin of the patient. In other embodiments, the pressure-applying device 290 can include an electrical pump and an inflatable cuff configured to generate external pressure against the optical sensor(s) 260. The electrical pump may inflate the cuff gradually causing the optical sensor(s) 260 to apply gradually increasing pressure to the skin of the patient.

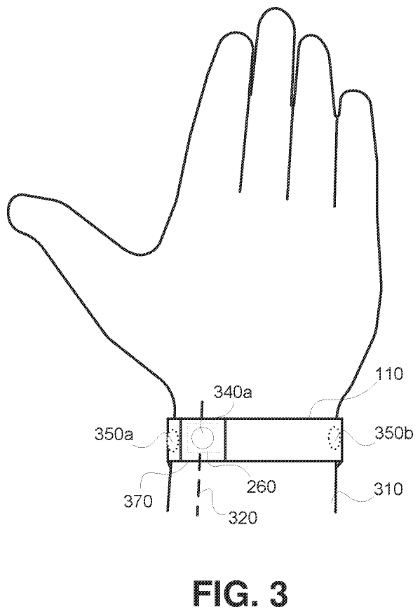

FIG. 3 is a block diagram illustrating an example wearable device 110 placed around a wrist of a patient. In the example of FIG. 3, the wearable device 110 is carried out in a shape of a watch, a ring, and/or a bracelet.

The electrical sensor 270 can include a differential amplifier operable to measure the electrical signal from the wrist. The electrical sensor 270 can include two or more active amplifier input plates embedded in the wearable device at opposite ends. For example, input plates 350a and 350b can be placed in contact with, respectively, the left and right sides of the wrist 310. Alternatively, or additionally, two input plates can be placed on opposite sides of the wearable device 110. In some embodiments, the first input plate 340a can be placed on the outer side the wearable device. The second input plate can be placed on the inner side of the wearable device. The second input plate can be in contact with the skin of the patient when the patient wears the wearable device. In some embodiments, the first input plate 340a can be placed in an area 370 of the wearable device 110. The area 270 may cover the radial artery 320 of a patient. The optical sensor(s) 260 can be placed on inner side of the area 370 of the wearable device 110.

In some embodiments, the optical sensor(s) 260 can be placed beneath a pulsating artery travelling along the arm and into a wrist 310. In some embodiments, a radial artery 320 passing in the inner wrist is used for measurements by the optical sensor(s) 260. In other embodiments, other arteries such as the ulnar artery, may be used. An external light source generating constant lighting can be used to radiate the pulsating artery. A beam reflected from the pulsating artery can be intercepted by the optical sensor(s) 260. In certain embodiments, a light of isosbestic wavelength is used to radiate the pulsating artery.

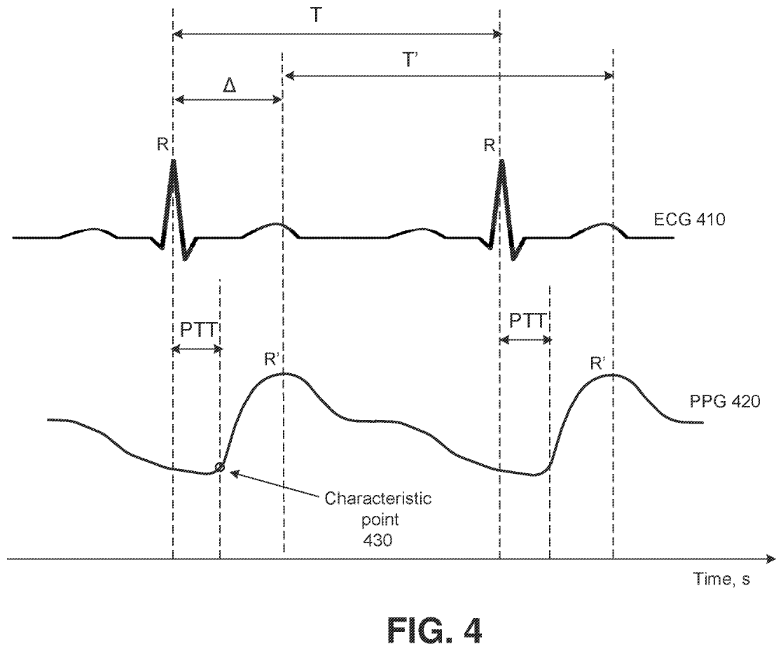

FIG. 4 shows plots of an example an example plot of an ECG 410, and an example plot of a PPG 420. The ECG 410 can be recorded with electrical sensor 270 using input plates placed on the wearable device 110. The ECG 410 can include R peaks corresponding to heart beats. Taking measurements from a single hand or a single wrist is challenging because the difference in voltages between measured locations is miniscule. The electrical signal measured at the wrist can include an ECG 410 and a noise. The noise can be caused by muscle activity, patient movements, and so forth. The noise component can be larger than the ECG. In some embodiments, the signal-to-noise ratio (SNR) is in the range of −40 dB to −60 dB. An example method for measuring a “clean” ECG from a wrist is described in U.S. patent application Ser. No. 14/738,666, titled “Wearable Device Electrocardiogram,” filed on Jun. 12, 2015.

The PPG 420 can be obtained by sensing a change in the color of skin. The change of the skin color is caused by a blood flow in a pulsating artery. In some embodiments, the PPG 420 can include peaks R′ related to the heart beats. Since it takes a time for blood to flow from the heart to the wrist, the peaks R′ are shifted by time periods Δ relative to the heart beats R in ECG 420. In some embodiments, shifts Δ can be measured as shift of a waveform of PPG (complex of PPG corresponding to period T′ in FIG. 4) relative to a waveform of ECG (complex of ECG corresponding to period T in FIG. 4).

In various embodiments, ECG 410 and PPG 420 are used to estimate a PTT. In some embodiments, PTT is defined as a time interval between the R peak in ECG 410 and characteristic point 430 located at the bottom of the PPG 420. PTT is a parameter that inversely correlates to BP. PTT decreases as BP increases, and PTT increases as BP decreases. Therefore, PTT can be used to estimate BP. In some embodiments, a regression equation can be derived to establish a relation between PTT and BP. The regression equation can be established for both systolic BP and diastolic BP. Alternatively, in other embodiments, other mathematical models, such as neural networks, may be used to establish the relation between the PTT and BP.

The location of characteristic point 430 can be uncertain or hard to detect. For example, a shape of PPG at a foot can be diffused when a pulse rate is high. Therefore, in some embodiments, when location of characteristic point 430 is uncertain or hard to detect, shifts between specific features of the ECG and PPG (such as certain landmarks or peaks) corresponding to the same heartbeat are used as an estimate for PTT. In certain embodiments, PTT is estimated based on shifts between waveforms of ECG and PPG corresponding to the same heartbeat.

PTT depends on the shape and cross-section area of a blood vessel (for example, a pulsating artery at which measurement is performed) since speed of blood travelling through the blood vessel depends on the cross-section area of the blood vessel and blood pressure.

According to various embodiments of the present disclosure, ECG and PPG are used to estimate PTT, PR, and diameter of the blood vessel or a change in the diameter of the blood vessel. In some embodiments, PTT, PR, and the diameter of the blood vessel or the change in the diameter of the blood vessel are then used to estimate BP. In some embodiments, PTT is determined based on ECG and PPG. PR can be found using a time period between two consecutive peaks in ECG or two consecutive peaks in PPG. In some embodiments, the diameter of the blood vessel or the change in the diameter of the blood vessel can be estimated using PPG.

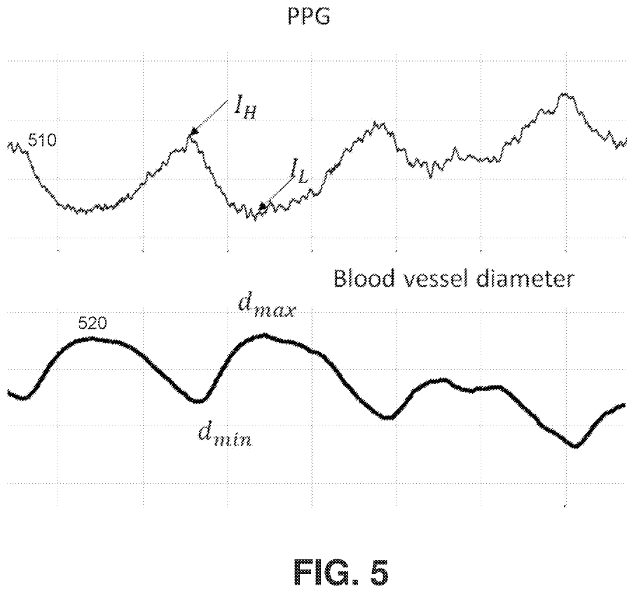

FIG. 5 shows an example plot of PPG 510 and an example plot of blood vessel diameter 520. The PPG 510 represents the intensity I of the light signal as modulated by a human tissue mostly due to a blood flow in the blood vessel. The high peaks (maximums) I_{H} of PPG 510 correspond to the low peaks dₘᵢₙ of the blood vessel diameter 520, and the low peaks I_{L} of the PPG 510 correspond to the high peaks dₘₐₓ of the blood vessel diameter 520.

In some embodiments, the detected PPG signal I, which is the intensity of light signal reflected from pulsating tissue, is modeled as follows:

I(t)=I₀*F*e^{-c*d(t)} (1).

In formula (1), I₀ represents an incident light intensity, F is indicates the absorption by pulsatile tissue, d(t) represents (arterial) blood vessel diameter, and c is overall absorption coefficient of blood hemoglobin derived from a mixture of both oxygen-saturated and non-oxygen saturated hemoglobin. Each of oxygen-saturated and non-oxygen saturated hemoglobin has its own particular value of absorption coefficient c for a particular wavelength of emitted light. Therefore, according to some embodiments, a light of isosbestic wavelength is used to radiate the pulsatile tissue allowing absorption coefficient c so it remains constant and independent of SpO2 oxygen saturation. The light absorption at the isosbestic wavelength is independent of SpO2 oxygen saturation because when a light of an isosbestic wavelength is used, the reflection from the oxygenized blood is the same as reflection from the non-oxygenized blood. In some embodiments, the isosbestic wavelength includes a near infrared wavelength 810 nm (NIR) and a green wavelength 520 nm (green). The NIR wavelength is more suitable for deeper vessels as it has deeper penetration while the green wavelength is more suitable for shallow vessels.

As shown in FIG. 5, the blood vessel diameter 520 changes periodically with the rhythm of the heart rate. The low peaks of the blood vessel diameter dₘᵢₙ correspond to the minimums of the absorption of the light by the blood and the high peaks of the light intensity I_{H}. The high peaks of the blood vessel diameter dₘₐₓ correspond to maximum absorption of the light by blood and the lowest peaks of the light intensity I_{L}. In some embodiments, the low peaks of the blood vessel diameter dₘᵢₙ can be considered to be constant as they reflect lowest diastole. The high peaks of the blood vessel diameter dₘₐₓ may vary relatively slowly due to, for example, fluctuations of blood pressure.

In some embodiments, it can be assumed that

I(t)≈I₀*F*(1−c*d(t)) (2).

Denoting further direct current (DC) component of PPG

DC=I₀*F (3)

and alternative current (AC) component

AC=I₀*F*c*d(t) (4),

an equation for determining blood vessel diameter d(t) can be written as:

$$( \frac{AC}{DC} ) = c * d ( t ) . \qquad ( 5 )$$

In equation (5), the AC component and DC component are found from PPG and absorption coefficient c is known. In some embodiments, change d(t)ₘₐₓ−d(t)ₘᵢₙ is used to estimate BP.

In other embodiments, BP is calculated from measured PTT, PR, and the diameter of the blood vessel or a change thereof using a pre-defined model. The pre-defined model describes a relationship between PTT, PR, and the diameter of the blood vessel and BP. In some embodiments, the pre-defined model is determined using statistical data collected during a calibration process. During the calibration process, a patient can wear the wearable device 110 to measure PTT, PR, and the diameter of the blood vessel or a change in the diameter of the blood vessel. Simultaneously, BP can be measured using an external device (for example, a conventional device for BP measurement). The calibration can be performed once at first usage of the wearable device 110 by a particular patient, and requires at least a single simultaneous measurement by the wearable device 110 and the external device. In other embodiments, several simultaneous measurements should be made to calibrate the wearable device 110 in a range of blood pressure values. The range of blood pressure values can be achieved by taking measurements at either or all the following: different times (hours of a day), different physical states of a patient, and different emotional states of the patient. Alternatively, lowering or elevating the arm and taking local blood pressure at the wrist with both an external device and the wearable device 110 can provide an effective means for mapping the PTT, PR, and diameter of the blood vessel or a change in the diameter of the blood vessel to a wide range of blood pressure values.

In some embodiments, the pre-defined model includes a three-dimensional model, wherein PTT, PR and the diameter of the blood vessel or a change in the diameter of the blood vessel are explanatory variables and systolic blood pressure is a dependent variable. Similarly, another three-dimensional model can be used to establish mathematical relationships between PTT, PR and diameter of blood vessel or a change in the diameter of the blood vessel as explanatory variables and diastolic blood pressure as a dependent variable.

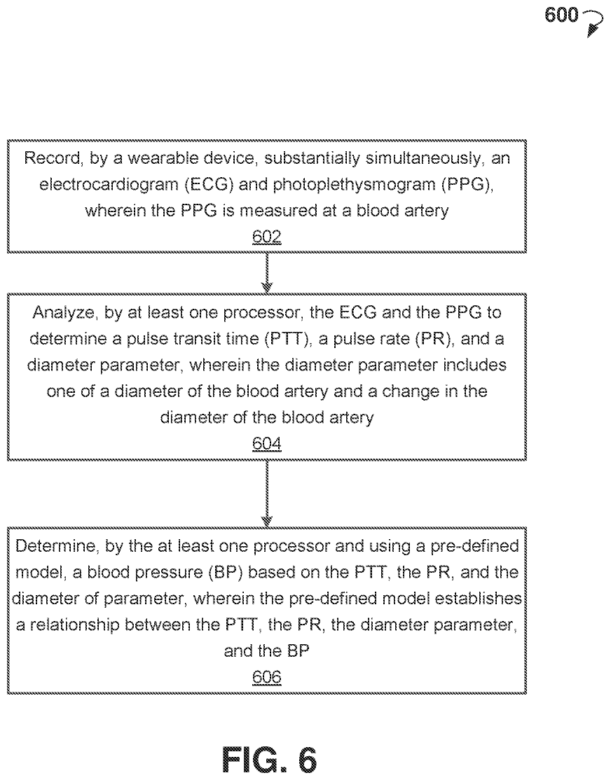

FIG. 6 is a flow chart showing steps of a method 600 for performing BP measurement, according to some embodiments. The method 600 can be implemented using wearable device 110 described in FIGS. 2 and 3 and system 100 described in FIG. 1. The method 600 may commence in block 602 with substantially simultaneous recording, by a wearable device, an ECG and a PPG. In some embodiments, PPG is measured at a blood artery. In some embodiments, ECG and PPG are recorded at a wrist.

In block 604, the method 600 proceeds with analyzing ECG and PPG to determine a PTT, a PR, and a diameter parameter. The diameter parameter may include a diameter of the blood artery or a change in the diameter of the blood artery. In block 606, the method 600 determines, based on PTT, PR, and the diameter parameter, BP using a pre-defined model. The pre-defined model establishes a relationship between the PTT, the PR, the diameter parameter, and the BP. In some embodiments, analysis of ECG and PPG and determination of PTT, the PR, the diameter parameter, and BP is performed locally using processor of the wearable device. In other embodiments, analysis of ECG and PPG and determination of PTT, the PR, the diameter parameter, and BP can be carried out remotely by a mobile device connected to the wearable device or in a computing cloud.

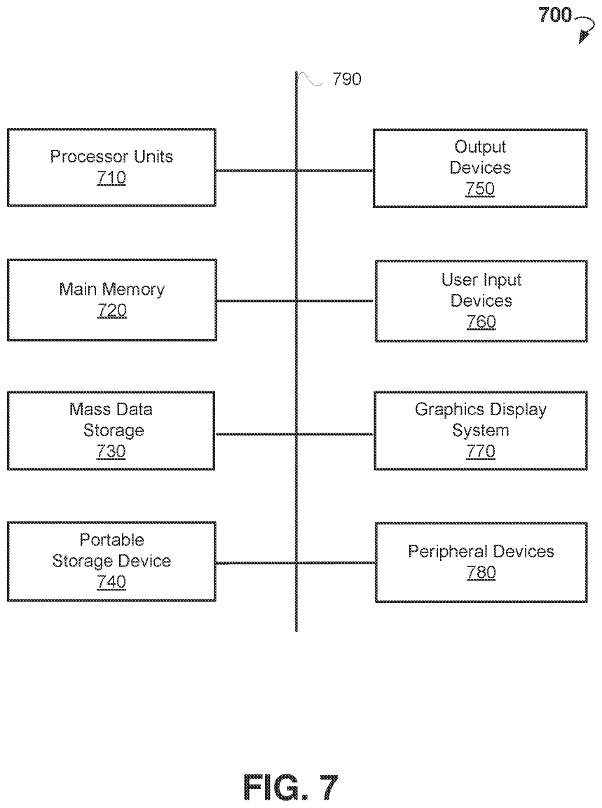

FIG. 7 illustrates a computer system 700 that may be used to implement embodiments of the present disclosure, according to an example embodiment. The computer system 700 may serve as a computing device for a machine, within which a set of instructions for causing the machine to perform any one or more of the methodologies discussed herein can be executed. The computer system 700 can be implemented in the contexts of the likes of computing systems, networks, servers, or combinations thereof. The computer system 700 includes one or more processor units 710 and main memory 720. Main memory 720 stores, in part, instructions and data for execution by processor units 710. Main memory 720 stores the executable code when in operation. The computer system 700 further includes a mass data storage 730, a portable storage device 740, output devices 750, user input devices 760, a graphics display system 770, and peripheral devices 780. The methods may be implemented in software that is cloud-based.

The components shown in FIG. 7 are depicted as being connected via a single bus 790. The components may be connected through one or more data transport means. Processor units 710 and main memory 720 are connected via a local microprocessor bus, and mass data storage 730, peripheral devices 780, the portable storage device 740, and graphics display system 770 are connected via one or more I/O buses.

Mass data storage 730, which can be implemented with a magnetic disk drive, solid state drive, or an optical disk drive, is a non-volatile storage device for storing data and instructions for use by processor units 710. Mass data storage 730 stores the system software for implementing embodiments of the present disclosure for purposes of loading that software into main memory 720.

The portable storage device 740 operates in conjunction with a portable non-volatile storage medium, such as a floppy disk, compact disk (CD), Digital Versatile Disc (DVD), or USB storage device, to input and output data and code to and from the computer system 700. The system software for implementing embodiments of the present disclosure is stored on such a portable medium and input to the computer system 700 via the portable storage device 740.

User input devices 760 provide a portion of a user interface. User input devices 760 include one or more microphones, an alphanumeric keypad, such as a keyboard, for inputting alphanumeric and other information, or a pointing device, such as a mouse, a trackball, stylus, or cursor direction keys. User input devices 760 can also include a touchscreen. Additionally, the computer system 700 includes output devices 750. Suitable output devices include speakers, printers, network interfaces, and monitors.

Graphics display system 770 includes a liquid crystal display or other suitable display device. Graphics display system 770 receives textual and graphical information and processes the information for output to the display device. Peripheral devices 780 may include any type of computer support device to add additional functionality to the computer system.

The components provided in the computer system 700 of FIG. 7 are those typically found in computer systems that may be suitable for use with embodiments of the present disclosure and are intended to represent a broad category of such computer components that are well known in the art. Thus, the computer system 700 can be a personal computer, handheld computing system, telephone, mobile computing system, workstation, tablet, phablet, mobile phone, server, minicomputer, mainframe computer, or any other computing system. The computer may also include different bus configurations, networked platforms, multi-processor platforms, and the like. Various operating systems may be used including UNIX, LINUX, WINDOWS, MAC OS, PALM OS, ANDROID, IOS, QNX, TIZEN and other suitable operating systems.

It is noteworthy that any hardware platform suitable for performing the processing described herein is suitable for use with the embodiments provided herein. Computer-readable storage media refer to any medium or media that participate in providing instructions to a central processing unit, a processor, a microcontroller, or the like. Such media may take forms including, but not limited to, non-volatile and volatile media such as optical or magnetic disks and dynamic memory, respectively. Common forms of computer-readable storage media include a floppy disk, a flexible disk, a hard disk, magnetic tape, any other magnetic storage medium, a CD Read Only Memory disk, DVD, Blu-ray disc, any other optical storage medium, RAM, Programmable Read-Only Memory, Erasable Programmable Read-Only Memory, Electronically Erasable Programmable Read-Only Memory, flash memory, and/or any other memory chip, module, or cartridge.

In some embodiments, the computer system 700 may be implemented as a cloud-based computing environment, such as a virtual machine operating within a computing cloud. In other embodiments, the computer system 700 may itself include a cloud-based computing environment, where the functionalities of the computer system 700 are executed in a distributed fashion. Thus, the computer system 700, when configured as a computing cloud, may include pluralities of computing devices in various forms, as will be described in greater detail below.

In general, a cloud-based computing environment is a resource that typically combines the computational power of a large grouping of processors (such as within web servers) and/or that combines the storage capacity of a large grouping of computer memories or storage devices. Systems that provide cloud-based resources may be utilized exclusively by their owners or such systems may be accessible to outside users who deploy applications within the computing infrastructure to obtain the benefit of large computational or storage resources.

The cloud may be formed, for example, by a network of web servers that comprise one or more computing devices, such as the computer system 700, with each server (or at least a plurality thereof) providing processor and/or storage resources. These servers may manage workloads provided by multiple users (e.g., cloud resource customers or other users). Typically, each user places workload demands upon the cloud that vary in real-time, sometimes dramatically. The nature and extent of these variations typically depends on the type of business associated with the user.

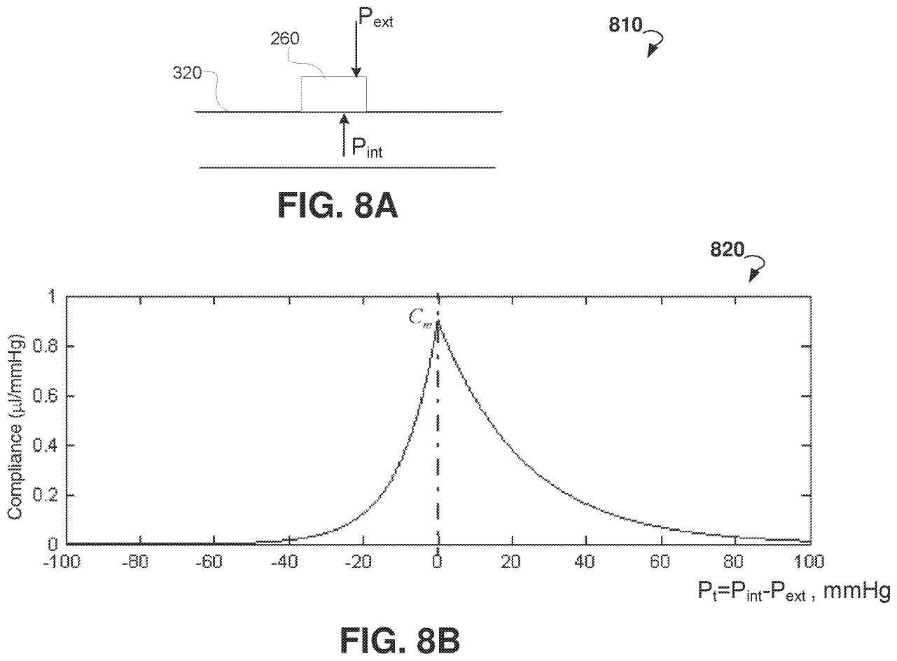

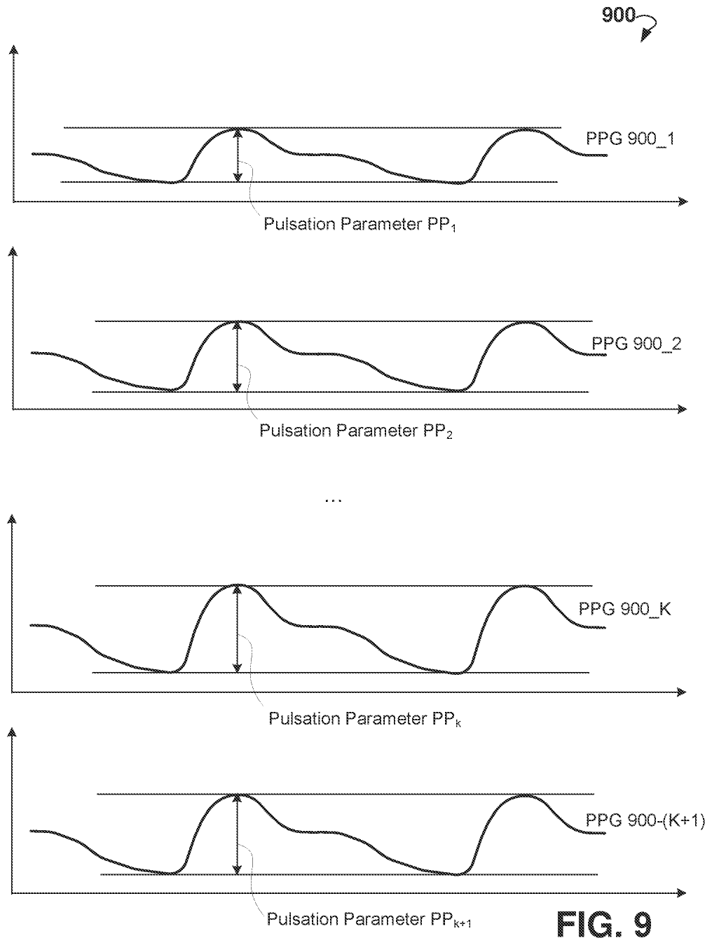

FIG. 8A is diagrammatic representation of a blood vessel 320 and optical sensor(s) 260. The optical sensor(s) 260 applies pressure Pₑₓₜ to the blood vessel 320. Pᵢₙₜ denotes mean intra-arterial pressure in the blood vessel 320. Determination of the PTT and the BP depend on the accuracy of determination of fluctuation Δd(t) of the blood vessel diameter d(t). The accuracy of determination of fluctuation Δd(t) of the blood vessel diameter d(t) can be contaminated due to either excessive or insufficient amount of the external pressure Pₑₓₜ applied to the blood vessel by the optical sensor(s) 260. Some values of external pressure Pₑₓₜ applied to the blood vessel may result in up to 5% error in PTT and up to 10% in BP.

The fluctuation of the Δd(t) of the blood vessel diameter d(t) depends on the properties of the blood vessel, specifically on compliance C. Compliance C can be determined as follows:

$$C = \frac{Δ V}{Δ P} , \qquad ( 6 )$$

where ΔV is the change of local volume of the blood vessel in response to change ΔP of distending pressure.

FIG. 8B is a plot of compliance C of a blood vessel. The value of the compliance C depends on value of transmural pressure Pₜ. The transmural pressure Pₜ is defined as a difference between the mean intra-arterial pressure Pᵢₙₜ and the external pressure Pₑₓₜ:

Pₜ=Pᵢₙₜ−Pₑₓₜ (7).

As shown in FIG. 8B, the compliance C reaches a maximum value at Pₜ=0. At the maximum value of compliance C the fluctuation of blood vessel volume ΔV (and, correspondently, the fluctuation Δd(t) of the blood vessel diameter d(t)) is maximum. If the external pressure Pₑₓₜ exceeds the mean intra-arterial pressure Pᵢₙₜ or the external pressure Pₑₓₜ is less than the mean intra-arterial pressure Pᵢₙₜ, then the compliance C is not maximum. In these situations, the fluctuation of blood vessel volume ΔV is not maximum.

As shown in FIG. 5, the fluctuation of the PPG 510 correlates with the fluctuation of the blood vessel diameter. Accordingly, at the maximum value of the compliance C, the fluctuation of the PPG 510 is also maximum. This fact can be used to determine a value of sensor pressure corresponding to the maximum value of the compliance C of the blood vessel.

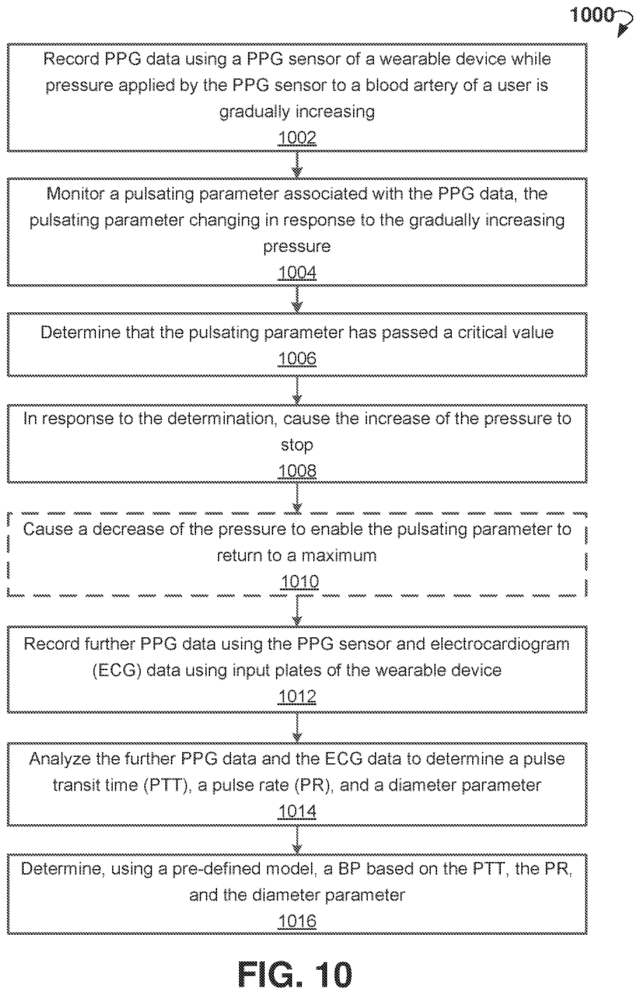

FIG. 9 shows plots of PPGs 900_k measured at different values P_{ext_k} of a pressure of an optical sensor(s) 260, according to some example embodiments. In these embodiments, the different values P_{ext_k} of the pressure applied by the optical sensor(s) 260 to the blood vessel can be applied manually by a patient. The patient can be prompted to gradually apply pressure to the optical sensor(s) 260 by using a finger of the other hand at the area 370 of the wearable device 110. As shown in FIG. 3 the area 370 may cover the blood vessel on the wrist of the patient, for example, the radial artery 320. In some embodiment, the patient can be instructed to touch the input plate 340a of the electrical sensor 270 to allow recording two-hand ECG at the same time.

The processor of the wearable device 110 may record, using the optical sensor(s) 260, the PPGs 900_k. For each of the PPGs 900_k, the processor can determine a pulsation parameter PPk. In some embodiments, the pulsation parameter PPk can include a difference between maximums and minimums of the PPGs 900_k. The processor of the wearable device 110 may monitor the change of the pulsation parameter PPk while the pressure P_{ext_k} increases gradually. The processor may determine that the pulsation parameter PPk has passed a critical value, for example, a maximum. For example, the processor may determine that the pulsating parameter has stopped increasing and started decreasing. Correspondently, after the pulsation parameter PPk has passed the critical value, the processor may instruct the patient to stop increasing the pressure on the optical sensor(s) 260. For example, the processor may cause the haptic device 270 to apply a force or vibration to the skin of the patient. Alternatively, the processor may cause the audio device 280 to generate a sound. In some embodiments, the patient may start decreasing the pressure on the optical sensor(s) 260 to allow the pulsating parameter to return to the maximum. The processor may determine that the pulsating parameter has returned to the maximum and instruct the patient to stop decreasing the pressure. For example, the processor may cause the haptic device 270 to vibrate the skin of the patient. The pattern of such vibration can be different from the pattern of the vibration used to prompt the patient to stop increasing the pressure. Alternatively, the processor may cause the audio device 280 to generate a sound. The frequency of the sound can be different from the frequency of the sound used to prompt the patient to stop increasing the pressure.

In other embodiments, the different values P_{ext_k} of the pressure applied by the optical sensor(s) 260 to blood vessel can be created automatically by the pressure-applying device 290. The processor may cause the pressure-applying device 290 to stop the increase in the pressure after the pulsating parameter has passed the critical value. For example, the processor may cause the pressure-applying device 290 to stop the increase in the pressure after determining that the pulsating parameter has stopped increasing and started decreasing, that the pulsating parameter has passed the maximum. The processor may cause the pressure-applying device 290 to decrease the pressure applied by the optical sensor(s) 260 to blood vessel to allow the pulsating parameter to return to the maximum.

After determining that the pulsating parameter has returned to the maximum, the processor may proceed with blood pressure measurements using, for example, method 600 described above with reference to FIG. 6. At these conditions, compliance C of the blood vessel is maximum. Accordingly, the fluctuation of blood vessel volume ΔV and, correspondently, the fluctuation Δd(t) of the blood vessel diameter d(t)) is maximum. Therefore, the errors in estimates of diameter parameter (d(t)ₘₐₓ−d(t)ₘᵢₙ), BP, and PTT are minimum.

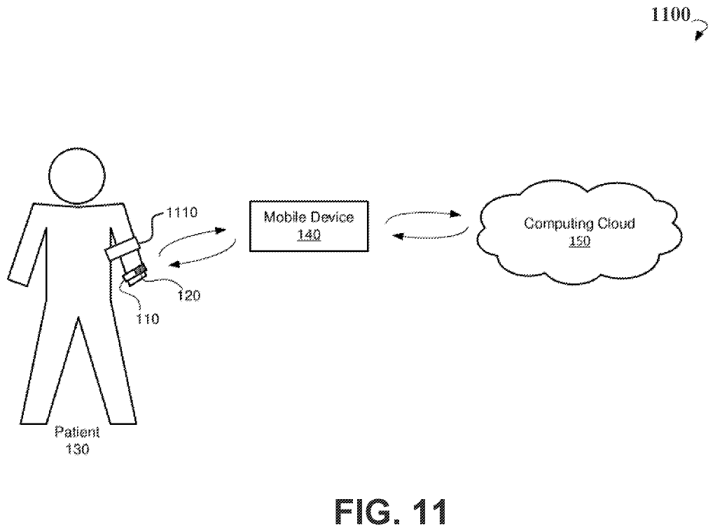

FIG. 10 is a flow chart showing an example method for optimizing sensor pressure in blood pressure measurements, according to some example embodiments. The method 1000 can be implemented using wearable device 110 described with reference to FIGS. 2 and 3 and system 100 described with reference to FIG. 1.

The method 1000 may commence in block 1002 with recording, by at least one processor, PPG) data using a PPG sensor of a wearable device while the pressure applied by the PPG sensor to a blood artery of the user is gradually increasing. The blood artery can be a radial artery of a wrist.

In block 1004, the method 1000 may monitor, by the processor, a pulsating parameter associated with the PPG data. The pulsating parameter may change in response to the gradually increasing pressure and include a difference between a maximum of the PPG data and a minimum of the PPG data.

In block 1006, the method 100 may determine, by the processor, that the pulsating parameter has passed a critical value. The determination that the pulsating parameter has passed the critical value may include determining that the pulsating parameter has stopped increasing and started decreasing. This may indicate that the pulsating parameter has passed the maximum.

The wearable device may include a pressure applying device configured to gradually apply an external pressure to the PPG sensor. Alternatively, the pressure can be increased by the user gradually applying external pressure to the PPG sensor.

In block 1008, in response to the determination that the pulsating parameter has passed the critical value, the method 1000 may stop increasing the pressure. The wearable device may include an alarm unit configured to prompt the user to stop applying the external pressure after the pulsating parameter has passed the critical value. The alarm unit may include a haptic device. The alarm unit may include a sound generating device.

In optional block 1010, the method 1000 may include causing, by processor, a decrease in the pressure to allow the pulsating parameter to return to the maximum. If the pressure is created by the user applying external pressure to the PPG sensor, then the processor may prompt the user to start and stop decreasing the pressure using the alarm unit. If the pressure is created by the pressure applying device, the pressure applying device can decrease the external pressure on the PPG sensor until the pulsating parameter returns to the maximum.

In block 1012, the method 1000 may proceed with recording further PPG data using the PPG sensor and electrocardiogram (ECG) data using input plates of the wearable device. In block 1014, the method 1000 may include analyzing, by the processor, further PPG data and ECG data to determine a pulse transit time (PTT), a pulse rate (PR), and a diameter parameter. The diameter parameter may include a change in the diameter of the blood artery.

Determination of the diameter parameter may include modifying the further PPG data by removing, from the further PPG data, an additive contribution resulting from a reflection of a light signal from a surface of a skin covering the blood artery and near-surface tissues underlying the skin and covering the blood artery. During the modification of the PPG data, a contribution resulting from the reflection of the light signal from the blood artery (contribution due to the reflection from the bulk blood volume) is kept unchanged. The additive contribution can be predetermined using a calibration process as described in U.S. patent application Ser. No. 14/738,711, titled “Pulse Oximetry,” filed on Jun. 12, 2015, incorporated herein by reference for all purposes.

The change in the diameter of the blood artery can be determined based on a ratio AC/DC, where AC is an alternating current component of the modified PPG data, and DC is a direct current component of the modified PPG data.

In block 1016, the method 1000 may include determining, by the processor and using a pre-defined model, a BP based on the PTT, the PR, and the diameter parameter. The pre-defined model can establish a relationship between the PTT, the PR, the diameter parameter, and the BP.

Thus, methods and systems for optimizing sensor pressure in blood pressure measurements using wearable devices have been described. Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

### Improved BP Measurements Incorporation PPG-Peak Monitoring

While the PTT and PPG signal can be used in a regression model to determine BP, including systolic, and diastolic and MAP (Mean Arterial Pressure) blood pressures, the accuracy of determining the BP and the BP as it changes over time can be improved by incorporating an additional variable in the BP determination. The additional variable is the PPG-pulse-peak values at the apex pressure point that occur during the cycles of applying pressure from the PPG sensor against an artery. For the purposes of this disclosure, the use of the phrase “applying pressure from the PPG sensor against an artery” includes the application of pressure by the PPG sensor to the skin over or substantially over the artery. The force that generates the pressure can be transmitted through a wearable device wristband or other material coupled to the PPG sensor. The PPG-pulse-peak values at the apex pressure point correspond to a MAP value. A baseline-MAP measurement can be used to convert the PPG-pulse-peak value to a MAP value. For the compensation of the parameters used in calculating a PTT value and initialization of a MAP model, a highly accurate initial baseline blood pressure and calculated baseline-MAP can be generated by a sphygmomanometer that includes a stethoscope and a blood pressure cuff.

The PPG-pulse-peak values and the resulting converted MAP value can be used to configure a MAP model. Further, the PPG-pulse-peak values and baseline blood pressure measurement can be incorporated as a correction factor into a pre-determined regression model based on calculated pulse-transit-times and a blood artery diameter parameter. In subsequent BP readings, the new PPG-pulse-peak values will be utilized with the MAP model or as incorporated into the pre-determined model correction factor parameter adjustment to provide a more accurate BP estimation.

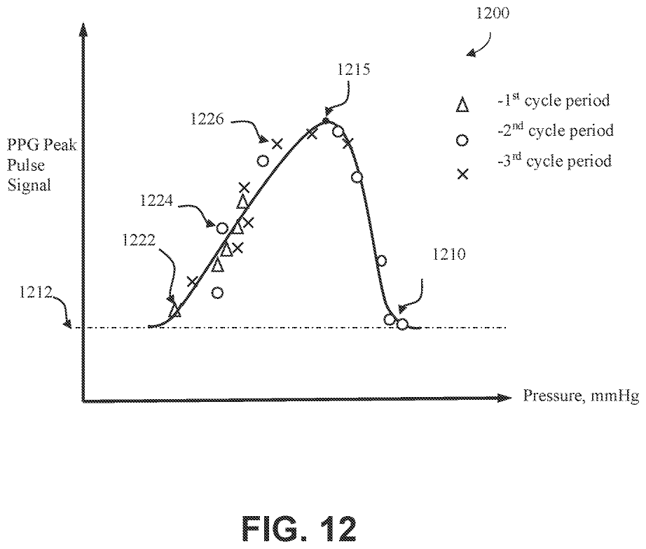

Referring now to FIG. 11, another example system 1100 for performing blood pressure measurements is shown. The system can include at least a wearable device 110. The wearable device 110 can include sensors 120. In some embodiments, the wearable device 110 is worn by a patient 130 (for example, on a wrist, ankle, earlobe, neck, chest, fingertip, and the like) for an extended period of time. In various embodiments, the wearable device 110 can be carried out as a watch, a bracelet, a wristband, a belt, a neck band, and the like.

The wearable device 110 can be operable to constantly collect, via sensors 120, sensor data from a patient 130. Based on the sensor data, the wearable device 110 can be operable to provide PPG and ECG data. The PPG and ECG data can be further used to obtain further medical parameters (for example, pulse rate, pulse transition time, blood pressure, and so forth).

In some embodiments, system 1100 includes a mobile device 140. The mobile device 140 can be communicatively coupled to the wearable device 110. In various embodiments, the mobile device 140 is operable to communicate with the wearable device 110 via a wireless connection, for example, Wi-Fi, Bluetooth, Infrared (IR), and the like. The mobile device 140 can include a mobile phone, a smartphone, a phablet, a tablet computer, a notebook, and so forth. The mobile device 140 can be operable to receive the sensor data and analyze the sensor data to provide PPG data and ECG data.

In further embodiments, the system 1100 may include a cloud-based computing resource (also referred to as a computing cloud) 150. In some embodiments, the computing cloud 150 includes one or more server farms/clusters comprising a collection of computer servers and is co-located with network switches and/or routers. In certain embodiments, the mobile device 140 is communicatively coupled to the computing cloud 150. The mobile device 140 can be operable to send the sensor data to the computing cloud 150 for further analysis (for example, for extracting medical parameters and PPG sensor and storing the results). The computing cloud 150 can be operable to run one or more applications and to provide reports regarding a health status of the patient, based on trends in medical parameters over time. The system 1100 can include a means 1110 to determine a reliable baseline blood pressure. This means can include but is not limited to a sphygmomanometer that includes a stethoscope and a blood pressure cuff 1110 that can be utilized to determine a diastolic and a systolic blood pressure. These values can be used to calculate a baseline MAP (Mean Arterial Pressure) value using the MAP equation as described below. The means to determine a reliable baseline blood pressure may be operated by a person and the results manually or automatically input into the system for use in a calibration process of a MAP model, correction of parameters in a pre-determined model of PTT, or both.

PPG pulse data is from an optical sensor. The processing of the PPG optical sensor data includes determining a PPG-pulse-peak value within the recording or monitoring of repetitive cycles of pressure to the optical sensors against the wrist radial artery or other artery. This process is described in detail below. The determined PPG-pulse peak value is required to determine MAP model coefficients for the conversion of a PPG-pulse-peak value to a MAP value. The equation and the determined equation coefficients can be used in subsequent PPG pulse data peak measurements. This relationship, i.e., determination of the MAP model coefficients, is determined by performing a substantially contemporaneous and reliable baseline measurement of the diastolic and systolic blood pressure 1110, which is used to calculate a baseline MAP value. For example, a blood pressure cuff can be used to determine an accurate diastolic and systolic blood pressure and calculate a baseline MAP value.

The conversion between the PPG-pulse-peak pulse value and a baseline MAP value can be modeled by different equations. Some equations may be more accurate, while others are easier to calculate. These equations can include a linear relationship where the model provides a conversion from the PPG-pulse-peak value to a MAP value is provided by scaling and adding a constant. However, other polynomials and non-linear equations are considered. Using this reliable baseline MAP value and a contemporaneous generation of a PPG-pulse-peak value, the equation coefficients can be used for generating subsequent MAP values.

This process assumes that if the wearable device is taken off and put back on or shifted from the previously worn location, the PPG sensor is still substantially located over the same artery. Because the PPG measurement is based on the ratio of the amplitudes of the infrared and visible light signal, the method is relatively insensitive to the variation in PPG signal strength during a BP reading.

The process for determining a MAP (Mean Arterial Pressure) is based on various physiological characteristics and attributes of the PPG signal. The first characteristic is that the PPG signal reaches a maximum when the pressure on the wrist radial artery equals the MAP. The MAP equation is defined as:

$$M A P = \frac{2 * P d + P s}{3} , \qquad ( 8 )$$

where Ps is the systolic BP and Pd is the diastolic BP. Thus, if the pressure asserted against the radial artery is lower than the MAP, the signal peaks representing the pulse peaks in the PPG sensor signal will occur at a radial artery pressure value lower than the MAP. As the pressure on the radial artery increases, the PPG pulse signal will increase until a maximum PPG pulse signal is reached. The pressure on the radial artery will equal the MAP. Further pressure by the PPG sensor on the radial artery will cause further restriction of the artery and reduce the pulse peaks in the PPG signal. When sufficient pressure is applied to the artery, the artery becomes occluded, and the pulses in the PPG signal will reach a minimum. A weak signal pulse may be present from the blood flow in the surrounding tissue. As pressure against the radial artery is reduced, this process is reversed.

The second important attribute of the PPG signal is that its amplitude can be used to set a baseline MAP value and be used to determine equation coefficients to convert the measured and processed PPG pulse data values to determine a MAP value in subsequent MAP measurements.

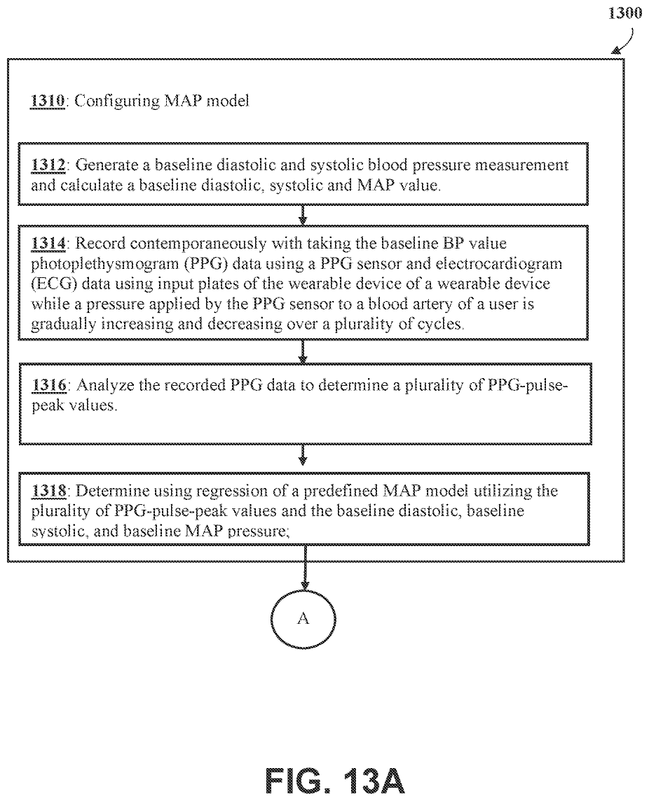

A plot of the PPG pulse amplitude vs. pressure on the radial artery can be represented by an asymmetric “bell” shaped or an asymmetric inverted paraboloid curve where the sensor pressure at the maximum PPG pulse value is the MAP value. The curve is typically asymmetric because of the physiology of the artery. Past testing has found the curve slope at pressures below the MAP peak pressure is less than the curve slope at pressures greater than the MAP peak. These different curve slopes before and after the MAP peak result in the asymmetrical shape of the curve. A representative curve of the PPG pulse values vs. Pressure against the radial artery is shown in FIG. 12.

As shown in FIG. 12, plot 1200 represents the PPG values 1222, 1224, 1226 from three cycles of asserting pressure on the wristband 110. The PPG values are taken from the peaks of the PPG signal, which corresponds to the systolic phase of a heartbeat. The pressure value at the curve peak or apex 1215 correlates to an estimated MAP value.

The triangular identified values, “Δ” 1222 show a cycle where the pressure on the wearable device wristband is not sufficient to exceed the apex pressure point 1215 where the PPG signal starts to decline.

The circular identified values, “O” 1224 show a cycle where the pressure on the wearable device wristband exceeds the PPG peak value 1215 and reaches the pressure point 1218 where the arterial blood flow is occluded and no systolic peaks by the PPG sensor are detected.

The values identified by a cross “X” 1226 show a cycle where the external pressures against the artery exceeds the peak 1215 point, but the pressure does not reach sufficient arterial pressure to cut off the arterial blood flow.

Thus, what can be expected from the PPG data is a rising of PPG signal during a pressure cycle, the PPG signal dropping off after an apex, and then reversing the cycle as the pressure is released.

Another physiological characteristic of the PPG signal is its relationship to the diastolic and systolic pressure in an artery. The heartbeat and associated PPG pulse peaks are typically spaced at intervals of between 0.5 seconds and 1.5 seconds apart. Thus, if the pressure from a wearable device is applied to the radial artery too quickly, the applied pressure that generates the PPG pulse peak value is likely missed. Since it is possible that there is not a PPG pulse peak and corresponding PPG data sample at the peak pressure, processing in a way of interpolation of the PPG pulse data can be used to estimate the PPG peak value.

The pressure asserted against the artery is similar to the pressure asserted by a blood pressure cuff. When the pulse amplitude of the PPG signal starts to rise above a threshold value or SNR (signal-to-noise ratio) in response to increasing pressure on an artery, this PPG signal value corresponds to the diastolic pressure. This pressure corresponds to the cuff pressure when a heartbeat is first heard, the diastolic pressure. This also corresponds to the point when the cuff pressure is being released and the heartbeat is no longer audible.

As the pressure against the artery increases beyond a peak value, the artery gradually becomes occluded until the arterial blood flow is completely cut off. Accordingly, the PPG signal gradually weakens to a point where the PPG pulse signal is below the noise level and is no longer ascertainable, or the SNR is low. This corresponds to the high pressure in a blood pressure cuff where the heartbeat is no longer audible, which represents the systolic pressure. This is also the pressure where the heartbeat becomes audible after the cuff pressure is increased beyond the systolic pressure and the cuff pressure reduced to where the heartbeat just becomes audible. Note that the determined PPG-pulse-peak value must be converted to a MAP value using a mathematical relationship to define a model and using a reliable baseline MAP measurement to set the model parameters.

Because there is not a measurement of the actual pressure being asserted by the PPG sensor against the artery, the shape of the curves defined by the PPG-peak values for each cycle are not the same except for the general characteristic of being an bell-shaped. However, each peak of a PPG pulse can be curve fitted and used to estimate an apex. The apex is the PPG-pulse-peak value which could represent the MAP value.

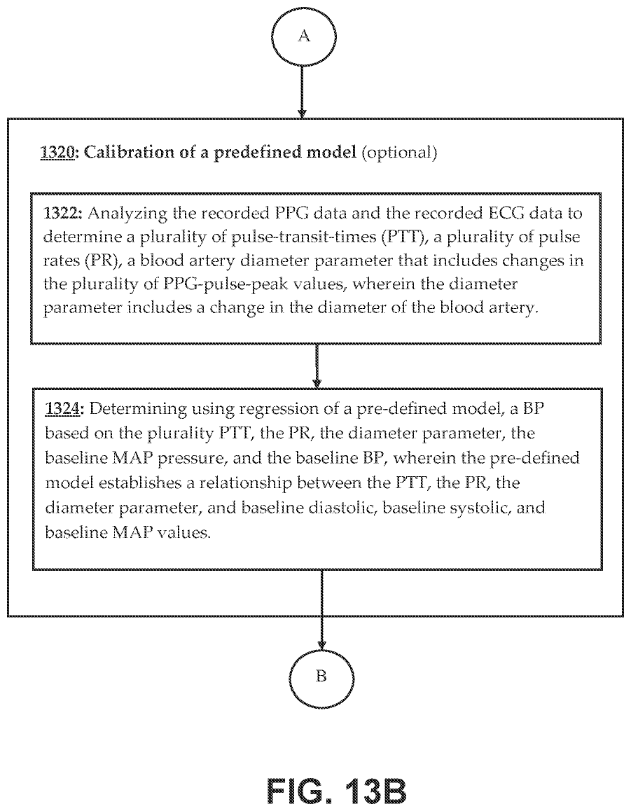

FIGS. 13A, 13B, and 13C disclose a process 1300 of using PPG data, ECG data, and PR (pulse rate) data from ECG plates, PPG sensor located over an artery and utilizing a reliable baseline MAP value to determine a MAP model to convert a PPG-peak-pulse value to a BP value that can include a diastolic, systolic, a MAP value or all of these. PPG data is recorded and processed during cycles of pressure against the artery by the PPG sensor. The PPG data is processed and the pulse peaks identified to determine one or more PPG-peak-pulse values.

The PPG component 250, 260 is comprised of LED lights 250 and an optical sensor 260. The optical sensor generates the PPG signal from which the PPG data is generated. The PPG calculated MAP value is calibrated from a reliable baseline diastolic and systolic BP measurement. The reliable baseline measurement can come from a blood pressure cuff device 1110.

For the reasons described above, the PPG-pulse-peak may not occur when the pressure being asserted by the PPG sensor, against the artery, corresponds with the true MAP pressure. Thus, the peaks of the PPG pulse data need to be curve fitted to determine an apex PPG value and thus the best estimate of the PPG-pulse-peak value. The increasing and decreasing pressure on the artery can be repeated for multiple cycles. Each pressure cycle can be curve-fitted, the PPG-pulse-peak-value (apex) estimated, and the estimated PPG-pulse-peak values averaged. This PPG-pulse-peak value can be used in conjunction with a MAP model and in conjunction with regression of a pre-determined model that utilizes PTT, pulse rates, and blood artery diameter parameter, MAP model, or the pressure determined by the new-MAP pressure. The MAP model can be an equation that has as an input the PPG-pulse-peak value and converts it to a MAP value.

This data can be stored in the wearable device 110 and transmitted or uploaded to a mobile device 140 that is in communication with the wearable device 110. The PPG data from the PPG sensor 260 can be uploaded to the mobile device 140 for processing the PPG data to find the PPG peak pulse value. Additionally, the conversion using a conversion equation and the conversion equation coefficients can generate a MAP value. The PPG data can be preprocessed for later transmission to the computing cloud 150. This PPG data can also be transmitted to a computing cloud 150 for storage, processing, display, and analysis with prior MAP equation data with other BP data acquired by other means, including but not limited to a blood pressure cuff.

In the processing steps of 1310, the MAP model is configured which can include parameter initialization. This process includes obtaining a baseline diastolic, systolic, and MAP value 1312, recording the PPG data through multiple cycles of pressing on an artery 1314, analyzing the PPG data to determine PPG-pulse peak values 1316, and determining a MAP model or equation coefficients for converting a PPG-pulse-peak value to a MAP value 1318.

In step 1312, a baseline MAP value is determined. A reliable measurement of the diastolic and systolic blood pressure is taken from a patient who has the wearable device 120. The diastolic and systolic values can be used to calculate a baseline MAP using the MAP equation. This measurement is required to determine coefficients for a conversion equation for converting a PPG peak value to a MAP value. The device can be a blood pressure cuff or sphygmomanometer 1110 but any other accurate means for measuring diastolic and systolic blood pressure can be used. The baseline diastolic and systolic pressures are used to compute a baseline MAP. The baseline diastolic and systolic pressures or baseline MAP or both are communicated to the wearable device or location where the calculations of new MAP values based on PPG-pulse-peak data are performed. Communication from the device that generates the baseline MAP value to where the processing occurs can be by any suitable means, including but not limited to manual entry or a wireless or wired communication means.

In step 1314, a contemporaneous recording of the PPG data is made to establish a baseline PPG-pulse-peak value. Contemporaneous, for the purpose of this invention, means within a period of time that one would not expect the health status to change for a person having their blood pressure monitored. For a person with stable health characteristics, contemporaneous means within five minutes of taking the baseline MAP. For a person whose health status may be changing more rapidly, contemporaneous means within a minute. In this step, PPG data is received from the PPG sensor on the wearable device.

This step includes providing an increasing and decreasing external force applied to the PPG sensor. The PPG sensor can be located on the wristband of a wearable device and is configured for the PPG sensor to be located perpendicular and over an artery, preferably the radial artery. The external force can be applied through the wristband to the PPG sensor.

To provide a better estimation of the PPG peak value, the external force is applied in one or more cycles. A cycle is the time from when the external pressure to the PPG sensor starts to when the external pressure is released or ends. Preferably, this cycle time is at least two seconds, but four to five seconds is better because more PPG pulses corresponding to heartbeats are recorded. Longer cycle times are contemplated. However, the longer the cycle, the harder it can be for a person to execute. Preferably, the cycle is repeated multiple times, and the external force is sufficient to reach a PPG peak pulse value. At least three cycles are preferable, but more cycles are contemplated by the invention.

In one embodiment, the system can provide feedback to adjust the application of the external force and when the PPG peak value has been achieved or passed. The feedback can be monitored by monitoring when different points in the asymmetrical curve shown in FIG. 12 are reached. For example, if the time between the start of the cycle and when an artery becomes occluded is too fast, one second, then feedback can be provided to slow the application of the external force. In another embodiment, the external force is provided by an automatic mechanical means.

In step 1316, the recorded PPG pulse data is analyzed as the peaks of the PPG pulse data change in response to the external force being gradually increased and released over each of the one or more cycles. In one embodiment, PPG pulses with the maximum value are determined and used to for curve fitting to generate a better estimate of the PPG-pulse-peak value. The analysis can be repeated for each cycle to generate one or more PPG-pulse-peak values.

In step 1318, the PPG-pulse-peak values are used to configure a MAP model or coefficients for a MAP equation. The data processing can validate each cycle based on the length of time of the cycle and whether it was detected that a peak value was passed. The shape of the asymmetrical bell curve can be used to help ascertain whether the PPG peak value was reached in a cycle. Some cycles can be excluded if the peak was not reached or the values deviate a large amount from the other cycles. A person of ordinary skill in the art of data processing would be able to provide an estimate of the PPG peak value using the PPG pulse data.

In one embodiment, the coefficients for conversion of the PPG-pulse-peak value to a MAP value can be a linear equation as shown below.

Baseline MAP=C0*(PPG peak value) (9)

The coefficient “C0” is determined for the equation using the data from the baseline MAP measurement and the contemporaneous determination of the PPG-pulse-peak values. Experimentation may determine that including a constant improves the accuracy of the PPG MAP measurement.

Baseline MAP=C0*(PPG peak value)+C1 (10)

Alternatively, non-linear formulation or a lookup table may be used for converting from a PPG-pulse-peak value to a MAP value.

In this optional processing step 1320, the baseline systolic, baseline diastolic, and baseline MAP can be used to improve the accuracy of a pre-determined model that utilizes ECG data, PPG data, pulse rate, and the diameter parameter. During this calibration step, an accurate BP is known from the previous accurate BP measurement step 1312. The PPG data and ECG data is analyzed 1322 to determine one or more pulse-transmission-time (PTT) and generated from the recorded PPG and ECG data. Regression of the pre-defined model using the one or more PPTs the PR, and the diameter parameter along with the baseline MAP values, the baseline diastolic, the baseline systolic or a combination thereof can establish the parameters for the relationship with the BP.

Next, in step 1330, the process waits for an indication that a new BP measurement is to be performed. This indication can be an input from a user or can be provided by an external timer or system management logic.

In step 1340, the process records further PPG pulse data and ECG data while pressure applied by the PPG sensor to a blood artery of a user is gradually increasing and decreasing over one or more cycles. As during calibration, the PPG sensor is still located substantially perpendicular to an artery.

In step 1350, the process analyzes the further-PPG data to determine one or more further PPG-pulse-peak values associated with the further-PPG data.

In step 1350, the PPG pulse data is monitored as the PPG pulse data changes in response to the external force being gradually increased and released over each of the one or more new cycles.

In step 1360, a new-MAP value is determined using the MAP model with the one or more further PPG-pulse-peak values. These further PPG-pulse-peak values can be estimated by the PPG peak values using curve fitting to determine the apex value, which corresponds to the MAP value.

In step 1370, the further-PPG data and the further-ECG are analyzed to determine one or more further PTTs, one or more further PRs based on the established relationship between the PTT, the PR, and the diameter parameter.

In step 1370, a new BP is determined using regression of the pre-defined model based on the plurality of further PTTs, the plurality of further PR, the blood artery diameter parameter that includes changes in the plurality of new PPG-pulse-peak values, wherein the pre-defined model establishes a relationship between the PTT, the PR, the diameter parameter, and the new-MAP value based on the MAP model.

The new-MAP value based on the MAP model can be included in the new BP in different ways. In one embodiment, a weighted average of the new-MAP value is made with the BP determined from the pre-defined model based on the plurality of further PTTs, the plurality of further PR, the blood artery diameter parameter that includes changes in the plurality of new PPG-pulse-peak values. The weighting can be equal or be weighted based on the previously determined accuracy of the system.

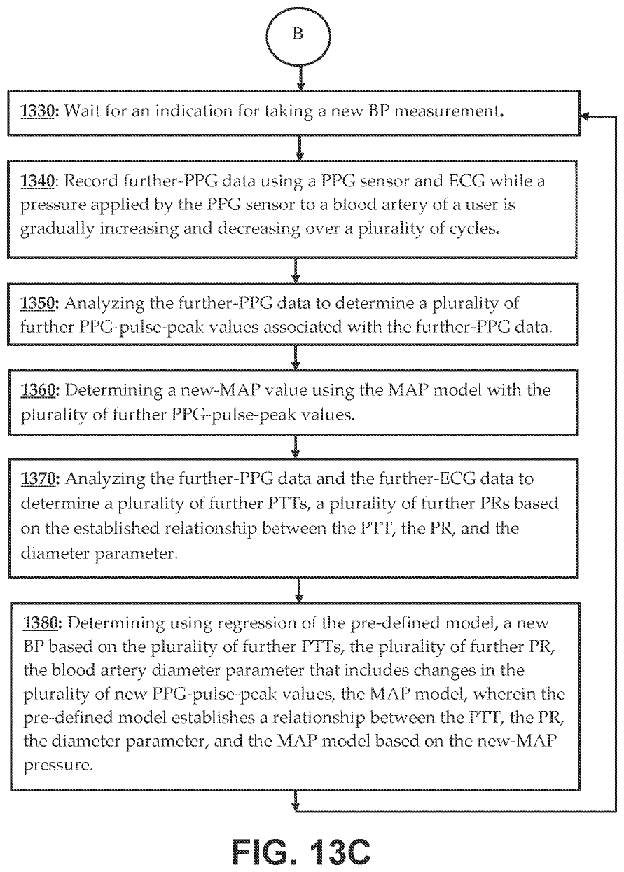

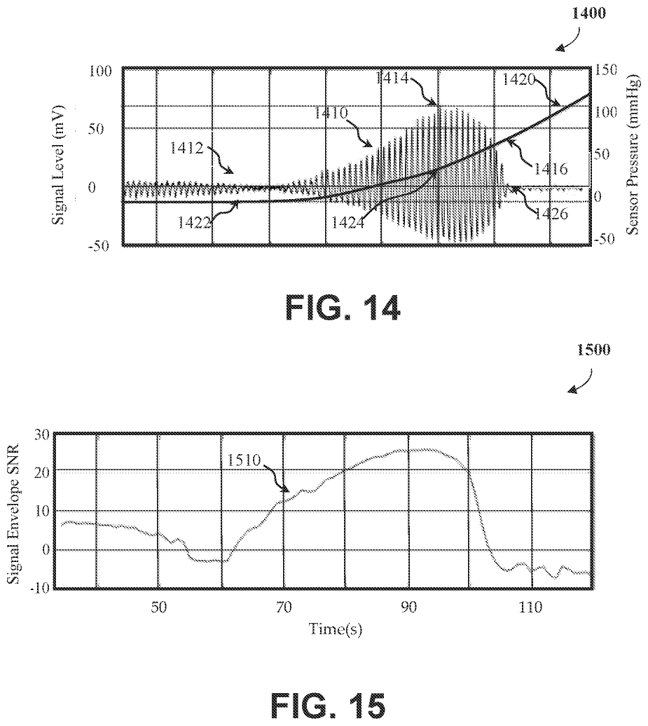

FIG. 14 and FIG. 15 are embodiments of test data showing the asymmetrical curve as shown in FIG. 12. FIG. 14 shows a graph 1400 of a recorded PPG signal 1410 over time as a function of increasing pressure 1420 between the PPG sensor and an area through which blood is flowing. This area can include an artery or finger. One difference is that the determined MAP at the finger does not exactly represent the mean pressure in the artery. The PPG signal starts with a low signal-to-noise level 1412 when no pressure is asserted 1422. As the pressure increases, the PPG signal 1414 reaches a maximum at the MAP pressure 1424. As the pressure is further increased 1426, the artery becomes occluded and the PPG pulse signal 1426 drops to the noise floor.

FIG. 15 shows a graph 1500 of the PPG signal envelope 1510 as pressure is applied. Note that the envelope 1510 has the same characteristics seen in FIG. 15. The value is low until pressure is applied. The envelope 1510 has a peak, and that the curve is asymmetrical.

Thus, methods and systems for using PPG data to generate a MAP measurement using wearable devices have been described. Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

What is claimed is:

1. A method for optimizing a blood pressure (BP) measurement, the BP measurement method comprising:
recording, by at least one processor, photoplethysmogram (PPG) data using a PPG sensor and electrocardiogram (ECG) data using input plates of a wearable device while a pressure applied by the PPG sensor to a blood artery of a user is gradually increasing and decreasing over one or more cycles;
analyzing, by the at least one processor, the recorded PPG data to determine one or more PPG-peak values associated with the recorded PPG data;
determining, by the at least one processor, and using regression of a predefined mean arterial pressure (MAP) model with the one or more PPG-peak values a MAP value;
analyzing, by the at least one processor, the PPG data and the ECG data to determine one or more PTTs, one or more PRs, a diameter parameter, wherein the diameter parameter includes a change in the diameter of the blood artery; and
determining, by the at least one processor and using regression of a pre-defined model, a BP based on the PTT, the PR, the blood artery diameter parameter that includes changes in the one or more PPG-peak values, wherein the pre-defined model establishes a relationship between the PTT, the PR, and the diameter parameter; and
determining an adjusted BP incorporating the MAP value and a baseline-MAP value.

2. The method of claim 1, further comprising determining a baseline-MAP value, the steps comprising:
measuring a baseline diastolic and a baseline systolic pressure;
calculating the baseline-MAP from the baseline diastolic and baseline systolic pressure;
recording contemporaneously with taking the baseline-MAP value, by the at least one processor, baseline-PPG data using the PPG sensor and baseline-ECG data using the input plates of the wearable device while a pressure applied by the PPG sensor to the blood artery of the user is gradually increasing and decreasing over one or more cycles;
analyzing, by the at least one processor, the recorded baseline-PPG data to determine one or more baseline-PPG-pulse-peak values; and
determining, by the at least one processor, and using regression of the predefined MAP model utilizing the one or more baseline-PPG-pulse-peak values the baseline MAP value.

3. The method of claim 1, wherein the generating a baseline-BP is performed with a blood pressure cuff.

4. The method of claim 1, wherein the pressure is increased and decreased by the user gradually applying and releasing an external pressure to the PPG sensor.

5. The method of claim 1, wherein the one or more cycles is at least three.

6. The method of claim 1, wherein each cycle of the one or more cycles is at least five seconds.

7. The method of claim 1, wherein the analyzing the recorded PPG data to determine one or more PPG-pulse peak values includes curve fitting peaks of the PPG data to determine the PPG-pulse peak values.

8. The method of claim 1, wherein the predefined MAP model is a linear equation.

9. The method of claim 1, wherein:
the determining the diameter parameter includes modifying the PPG data by removing, from the PPG data, an additive contribution resulting from a reflection of a light signal from a surface of a skin covering the blood artery and near-surface tissues underlying the skin and covering the blood artery and keeping, in the PPG data, a contribution resulting from the reflection of the light signal from the blood artery unchanged, the additive contribution being predetermined using a calibration process; and
the change in the diameter of the blood artery is determined based on a ratio AC/DC, wherein AC is an alternating current component of the modified PPG data, and DC is a direct current component of the modified PPG data.

10. A system optimizing a blood pressure (BP) measurement, the system comprising:
a wearable device including a photoplethysmogram (PPG) sensor and electrocardiogram (ECG) input plates; and
at least one processor communicatively coupled to the wearable device, the at least one processor being configured to perform the steps:
receiving a baseline MAP value;
recording, by at least one processor, photoplethysmogram (PPG) data using a PPG sensor and electrocardiogram (ECG) data using input plates of the wearable device while a pressure applied by the PPG sensor to a blood artery of a user is gradually increasing and decreasing over one or more cycles;
analyzing, by the at least one processor, the recorded PPG data to determine one or more PPG-peak values associated with the recorded PPG data;
determining, by the at least one processor, and using regression of a predefined mean arterial pressure (MAP) model with the one or more PPG-peak values a MAP value;
analyzing, by the at least one processor, the PPG data and the ECG data to determine one or more PTTs, one or more PRs, a diameter parameter, wherein the diameter parameter includes a change in the diameter of the blood artery; and
determining, by the at least one processor and using regression of a pre-defined model, a BP based on the PTT, the PR, the blood artery diameter parameter that includes changes in the one or more PPG-peak values, wherein the pre-defined model establishes a relationship between the PTT, the PR, and the diameter parameter; and
determining an adjusted BP incorporating the MAP value and a baseline-MAP value.

11. The system of claim 10, wherein determining a baseline-MAP value comprises the steps:
measuring a baseline diastolic and a baseline systolic pressure;
calculating the baseline-MAP from the baseline diastolic and baseline systolic pressure;
recording contemporaneously with taking the baseline-MAP value, by the at least one processor, baseline-PPG data using the PPG sensor and baseline-ECG data using the input plates of the wearable device while a pressure applied by the PPG sensor to the blood artery of the user is gradually increasing and decreasing over one or more cycles;
analyzing, by the at least one processor, the recorded baseline-PPG data to determine one or more baseline-PPG-pulse-peak values; and
determining, by the at least one processor, and using regression of the predefined MAP model utilizing the one or more baseline-PPG-pulse-peak values the baseline MAP value.

12. The system of claim 11, wherein the generating a baseline-BP is performed with a blood pressure cuff.

13. The system of claim 11, wherein the pressure is increased and decreased by the user gradually applying and releasing an external pressure to the PPG sensor.

14. The system of claim 11, wherein the one or more cycles is at least three.

15. The system of claim 11, wherein each cycle of the one or more cycles is at least five seconds.

16. The system of claim 11, wherein the analyzing the recorded PPG data to determine one or more PPG-pulse peak values includes curve fitting peaks of the PPG data to determine the PPG-pulse peak values.

17. The system of claim 11, wherein the predefined MAP model is a linear equation.

18. The system of claim 11, wherein the determining the diameter parameter includes modifying the further PPG data by removing, from the further PPG data, an additive contribution resulting from a reflection of a light signal from a surface of a skin covering the blood artery and near-surface tissues underlying the skin and covering the blood artery and keeping, in the PPG data, a contribution resulting from the reflection of the light signal from the blood artery unchanged, the additive contribution being predetermined using a calibration process; and
the change in the diameter of the blood artery is determined based on a ratio AC/DC, wherein AC is an alternating current component of the modified PPG data, and DC is a direct current component of the modified PPG data.

19. A non-transitory computer-readable storage medium having embodied thereon instructions, which when executed by at least one processor, perform steps of a method, the method comprising:
recording, by at least one processor, photoplethysmogram (PPG) data using a PPG sensor and electrocardiogram (ECG) data using input plates of a wearable device while a pressure applied by the PPG sensor to a blood artery of a user is gradually increasing and decreasing over one or more cycles;
analyzing, by the at least one processor, the recorded PPG data to determine one or more PPG-peak values associated with the recorded PPG data;
determining, by the at least one processor, and using regression of a predefined mean arterial pressure (MAP) model with the one or more PPG-peak values a MAP value;
analyzing, by the at least one processor, the PPG data and the ECG data to determine one or more PTTs, one or more PRs, a diameter parameter, wherein the diameter parameter includes a change in the diameter of the blood artery; and
determining, by the at least one processor and using regression of a pre-defined model, a BP based on the PTT, the PR, the blood artery diameter parameter that includes changes in the one or more PPG-peak values, wherein the pre-defined model establishes a relationship between the PTT, the PR, and the diameter parameter; and
determining an adjusted BP incorporating the MAP value and a baseline-MAP value.

20. The non-transitory computer-readable storage medium of claim 19, further comprising
determining a baseline-MAP value, the steps comprising:
measuring a baseline diastolic and a baseline systolic pressure;
calculating the baseline-MAP from the baseline diastolic and baseline systolic pressure;
recording contemporaneously with taking the baseline-MAP value, by the at least one processor, baseline-PPG data using the PPG sensor and baseline-ECG data using the input plates of the wearable device while a pressure applied by the PPG sensor to the blood artery of the user is gradually increasing and decreasing over one or more cycles;
analyzing, by the at least one processor, the recorded baseline-PPG data to determine one or more baseline-PPG-pulse-peak values; and
determining, by the at least one processor, and using regression of the predefined MAP model utilizing the one or more baseline-PPG-pulse-peak values the baseline MAP value.
